(19) United States
(12) Patent Application Publication
(10) Pub. No.: US 2024/0000700 A1
(43) Pub. Date: Jan. 4, 2024

(54) PLANT EXTRACTING METHOD

(71) Applicant: GIVAUDAN SA, VERNIER (CH)

(72) Inventors: Benedicte SENNELIER PORTET, Jonquerettes (FR); Marie MEUNIER, Les Petites Loges (FR); Romain REYNAUD, Toulouse (FR); Amandine SCANDOLERA, Sault-Saint-Remy (FR)

(21) Appl. No.: 18/253,813

(22) PCT Filed: Dec. 17, 2021
(86) PCT No.: PCT/EP2021/086606

§ 371 Date: May 22, 2023

(30) Foreign Application Priority Data
Dec. 18, 2020 (GB) 2020183.6

(51) Int. Cl. A61K 8/9789; A61Q 5/00

(52) U.S. Cl. CPC A61K 8/9789; A61K 2236/30; A61Q 5/006

(57) ABSTRACT

The present disclosure provides a method of preparing a cosmetic active ingredient from patchouli, as well as the cosmetic active ingredient thus obtained and cosmetic compositions comprising the same.

The present invention relates to a method of preparing a cosmetic active ingredient from exhausted patchouli aerial parts.

Patchouli (Pogostemon cablin) is a species of plant from the family Lamiaceae, commonly called the “mint” or “deadnettle” family. The plant grows as a bushy herb, with erect stems reaching around 75 cm in height and bearing small, pale pink-white flowers. It is native to tropical regions of Asia.

The heavy and strong scent of patchouli has been used for centuries in perfumes and, more recently, in incense, insect repellents, and alternative medicines. Pogostemon cablin, among other members of the Pogostemon genus, is commonly cultivated for its essential oil, known as patchouli oil. Patchouli grows well in warm to tropical climates. The seed-producing flowers are very fragrant and blossom in late fall. The tiny seeds may be harvested for planting, but they are very delicate and easily crushed. Cuttings from the mother plant can also be rooted in water to produce additional plants.

Extraction of patchouli's essential oil is typically achieved by steam distillation of the dried leaves and stems, requiring rupture of its cell walls by steam scalding, light fermentation, or drying. Leaves may be harvested several times a year and, when dried, may be exported for distillation.

Patchouli oil is used widely in fine fragrance and perfumery, and it is an important ingredient in East Asian incense. Patchouli leaves have been used to make an herbal tea. In some cultures, patchouli leaves are eaten as a vegetable or used as a seasoning. Patchouli has also been described as an insect repellent, in particular against the Formosan subterranean termite.

90% of the world's patchouli oil supply stems from Indonesia (about 1′000 to 1′200 Megatons per year). For the production of patchouli oil, patchouli stems and leaves—i.e. the aerial parts—are used. They are typically dried during a few days, then gathered into bundles and stored a few days before distillation. This storage allows a light fermentation process to release some key odour components of the plant. A first steam distillation is typically performed locally by the farmers, and the resulting oil may be re-distilled in a stainless steel vessel to provide a stable and colourless patchouli oil.

kg of dried aerial parts (obtained from about 200 kg of fresh aerial parts) provide about 1 kg of patchouli oil.

The patchouli aerial parts used for distillation typically consist of about 70% leaves and 30% stems (no flowers).

As a side product of the patchouli oil production, about 50 Megatons of “exhausted” patchouli aerial parts are formed, i.e. of residual patchouli aerial parts that have been subjected to steam distillation. As mentioned above, prior to the steam distillation, these patchouli aerial parts are usually dried and lightly fermented.

It is highly desirable that these exhausted patchouli aerial parts could be up-cycled and further used.

To this end, WO 2019/193203 A1 describes a patchouli stem extract prepared from exhausted patchouli stems, which may be used as a cosmetic active ingredient. The extract is obtained by extraction with water and/or alcohol.

### SUMMARY OF THE INVENTION

Surprisingly, it has now been found that it is possible to significantly improve the extraction process and to obtain a more efficient cosmetic active ingredient.

In a first aspect, the present invention relates to a method of preparing a cosmetic active ingredient by sub-critical water extraction on the exhausted patchouli aerial parts.

In a second aspect, the present invention relates to a cosmetic active ingredient obtained by said method.

In a third aspect, the present invention relates to a cosmetic composition comprising said cosmetic active ingredient.

In a fourth aspect, the present invention relates to a method of stimulating the sebum production and/or of reducing the formation of dry flakes by applying the cosmetic active ingredient or the cosmetic composition of the present invention to human scalp.

Details of the Invention

The present invention provides a method of preparing a cosmetic active ingredient, said method comprising the steps of:
a) providing exhausted patchouli aerial parts; and
b) performing a sub-critical water extraction on the exhausted patchouli aerial parts.

The method of the present invention involves an extraction with sub-critical water.

Sub-critical water—also known as “super-heated water” or “pressurized hot water”—is liquid water under pressure at temperatures between the usual boiling point, 100° C., and the critical temperature, 374° C., of water. Sub-critical water is stabilized by overpressure that raises the boiling point, or by heating it in a sealed vessel with a headspace, where the liquid water is in equilibrium with vapor at the saturated vapor pressure.

The term “exhausted patchouli aerial parts” will be used throughout this application. It refers to patchouli aerial parts that have previously been subjected to distillation, in particular to steam distillation. Optionally, the patchouli aerial parts may also have been dried and/or fermented.

The use of exhausted patchouli aerial parts allows for up-cycling the material, thereby significantly reducing waste.

Furthermore, the use of sub-critical water for the extraction renders the method of the present invention very eco-friendly and green: There is no toxicity and no residual solvent. Furthermore, sub-critical water is non-flammable.

Overall, the sub-critical water extraction used in the method of the present invention is COSMOS approved.

It has been found that, thanks to the use of sub-critical water, the extraction time could be shortened.

It has further been found that the extract obtained by the method of the present invention exhibits a higher sugar content and an increased cosmetic activity compared to extracts prepared by conventional methods. It is also fully natural.

The extract obtained by the method of the present invention typically has a dry matter content of about 1-5 wt %.

The exhausted patchouli aerial parts may be reduced to smaller pieces prior to the sub-critical water extraction, in particular crushed, cut and/or ground. For instance, the exhausted patchouli aerial parts may be reduced to a size of about 4-5 mm.

The exhausted patchouli aerial parts may also be washed prior to the extraction, for instance with water.

In an embodiment of the present invention, the sub-critical water extraction is performed at a temperature of 100 to 175° C., more preferably at a temperature of 110 to 150° C., most preferably at a temperature of about 125° C. It has been found that best results can be obtained in this temperature range.

In an embodiment of the present invention, the sub-critical water extraction is performed at a pressure of 1 to 50 bar more preferably at a pressure of 10 to 30 bar, most preferably at a pressure of about 20 bar. It has been found that best results can be obtained in this pressure range.

For example, the sub-critical water extraction may be performed at a temperature of about 125° C. and a pressure of about 20 bar.

After the sub-critical water extraction, the extract is typically brought to room temperature and ambient pressure. Cooling may be achieved by means of an ice or water bath, if desired.

In an embodiment, the method of the present invention further comprises the step of:
c) solid-liquid separation.

Solid-liquid separation may be achieved by filtration or centrifugation, for instance. Preferably, the extract is filtered. The solid-liquid separation may be performed directly in the extraction vessel, if it is suitably equipped.

In an embodiment, the method of the present invention further comprises the step of:
d) adding a preservative, preferably phenethyl alcohol.

In the context of the present invention, the term “preservative” is meant to cover any substance that is listed as a preservative in the European Cosmetics Regulation (Regulation (EC) N° 1223/2009 of the European Parliament and of the Council of 30 Nov. 2009 on Cosmetic Products, in particular Annex V), as well as any substance that may not be listed therein but is able to provide the same or at least a similar function. i.e. “preservative-like” substances.

Examples of suitable preservatives include, but are not limited to, potassium sorbate, sodium benzoate, salicylic acid, 1,2-hexanediol, caprylyl glycol, tropolone, ethylhexylglycerin, phenyl propanol, pentylene glycol, phenethyl alcohol (=2-phenylethanol), propanediol, and mixtures thereof, for example Symdiol® 68T (1,2-hexanediol, caprylyl glycol, tropolone) or Sensiva® PA 20 (phenethyl alcohol, ethylhexylglycerin, sodium benzoate). Preferred examples include phenyl propanol, phenethyl alcohol, and propanediol, in particular phenethyl alcohol.

It has been found that phenethyl alcohol is particularly preferred because it is Cosmos compliant, available in a natural origin version according to the 15012128 regulation, and has been found to provide effective antibacterial activity.

Suitable concentrations of phenethyl alcohol are from about 0.5 wt % to about 5 wt %, preferably from about 0.8 wt % to about 2 wt %, more preferably about 1 wt %.

The extract obtained by the method of the present invention may be purified, for example by filtration (e.g. on KDS15 filters), charcoal treatment and/or sterile filtration.

In particular, the extract may be filtered, e.g. after the addition of a preservative, if desired under vacuum. A suitable porosity is about 0.2 μm, for instance.

Alternatively or in addition, the extract may also be decolorized by the addition of a bleaching agent, such as bentonite, charcoal powder or granulate, bleaching earth or Tonsil 115 FF, or passed over a charcoal filter. Preferably, decoloration is done prior to the addition of a preservative.

Alternatively or in addition, the extract may also be sterilized, e.g. by sterile filtration or heat treatment (pasteurization).

In an embodiment, the method of the present invention further comprises the step of:
e) sterilization, preferably by sterile filtration.

The extract obtained by the method of the present invention may also be concentrated if desired. It is possible to add a solvent, e.g. 1,3-propanediol, pentylene glycol, butylene glycol, or propylene glycol, to the extract prior to concentration to improve the solubility of the extract during concentration. Preferably, these solvents are of natural origin.

Preferably, steps a), b), c), d) and e) are performed in this order.

Alternatively or in addition, the extract obtained by the method of the present invention may also be diluted, e.g. with a preferred solvent. For instance, water may be added to the extract. In an embodiment, the extract is diluted with water at a ratio of about 1:1.

In an embodiment, the cosmetic active ingredient of the present invention comprises about 1.0-4.99% of sub-critical patchouli extract (on a dry matter basis), about 1.0-4.99% of phenethyl alcohol, and at least 50% of water.

In an embodiment, the cosmetic active ingredient of the present invention consists of about 1.0-4.99% of sub-critical patchouli extract (on a dry matter basis), about 1.0-4.99% of phenethyl alcohol, and the remainder is water.

The present invention further provides a cosmetic active ingredient obtained by the method of the present invention.

It has been found that the extract obtained by the method of the present invention has a particularly high sugar content and provides desirable cosmetic activity.

In particular, it was found to exhibit antioxidant, anti-hyaluronidase, and anti-tyrosinase activities. Excessive oxidative stress can lead to skin damages, promoting scalp dryness and consequently dry dandruff.

The cosmetic active ingredient of the present invention was found to provide a stimulation of sebum production, a stimulation of antioxidant property and a strong inhibition of wound healing through a control of keratinocytes migration. In a previous study, it was found that the quantity of sebum has an impact on microbial diversity on scalp: Oily scalp exhibits a 718% higher sebum production than dry scalp; and the alpha diversity (Shannon index) is 25% lower in oily scalp than in dry scalp.

Thanks to the above activities, the cosmetic active ingredient of the present invention is ideal for treatment of dry dandruff and white flakes.

More details on the activity of the cosmetic active ingredient of the present invention are provided in the examples below.

The total sugar content on a dry basis may be determined by evaporating the extract to dryness, e.g. using a rotary evaporator, and then quantifying the amount of sugars (including mono-, oligo- and polysaccharides) on a weight per weight basis. Alternatively, the total sugar content may also be determined in the liquid extract and then calculated on a dry basis, taking into account the dry matter content of the extract. Quantification may be done using spectrophotometric methods (e.g. Dubois method), HPLC-ELSD or HPAE-PAD using a calibration curve of simple sugars (typically glucose equivalent).

It was found that the extract obtained by the method of the present invention (i.e. the cosmetic active ingredient of the present invention) has a particularly high sugar content, especially in comparison to extracts obtained by other methods.

In an embodiment of the present invention, the cosmetic active ingredient comprises a total sugar content of about 3-15 wt % on a dry basis, more preferably at least 10 wt %.

More specifically, a large amount of oligosaccharides and proteoglycans was observed.

The present invention further provides a cosmetic composition comprising the cosmetic active ingredient of the present invention and a carrier.

The carrier should be cosmetically acceptable, and in particular dermatologically acceptable.

The cosmetic composition according to the present invention may further comprise one or more materials selected from the group consisting of solvents, surfactants, thickeners, styling polymers, anti-dandruff actives, antimicrobial materials, skin and scalp actives, vitamins, salts, buffers, hair growth agents, conditioning materials, hair-fixative polymers, fragrances, colorings/colorants, dyes, pigments, opacifiers, pearlescent aids, oils, waxes, preservatives, sensates, sunscreens, medicinal agents, antifoaming agents, antioxidants, binders, biological additives, buffering agents, bulking agents, chelating agents, chemical additives, film formers or materials, pH adjusters, propellants, oxidizing agents, and reducing agents.

All additives should be physically and chemically compatible with the essential components of the cosmetic composition, and should not otherwise unduly impair stability, aesthetics or performance. Most importantly, they should also be cosmetically acceptable.

The cosmetic composition of the present invention comprises a carrier, which is typically present at a level of about 20 wt % to about 99 wt %. The carrier may comprise water, organic solvents (miscible or non-miscible with water) silicone solvents, and/or mixtures thereof. The solvents should be dermatologically acceptable. Carriers usually do not comprise more than about 2 wt % of non-volatile solvent, as significantly higher concentrations will increase hair weigh-down and greasy feel. Water, organic and silicone solvents that have boiling points below or equal to 250° C. are considered volatile solvents. Suitable carriers typically include water and water solutions of lower alkyl alcohols, such as monohydric alcohols having 1 to 6 carbons (e.g. ethanol and/or isopropanol), and polyhydric alcohols, such as glycols, glycerine, and other diols.

As thickeners, the cosmetic composition may comprise rheology modifiers to improve feel, in-use properties and suspending stability. For example, rheological properties may be adjusted so that the composition remains uniform during its storage and transportation and does not drip undesirably onto other areas of the body, clothing or home furnishings during its use. Any suitable rheology modifier can be used. Typically, about 0.01 to about 3 wt % of thickener is included. Examples of suitable thickeners are disclosed in WO 2015/035164 and US 2001/0043912, the contents of which in this respect are herewith incorporated by reference.

The cosmetic compositions of the present invention may also contain optional components, which modify the physical and performance characteristics. Such components include surfactants, salts, buffers, thickeners, solvents, opacifiers, pearlescent aids, preservatives, fragrances, colorants, dyes, pigments, chelators, sunscreens, vitamins, and medicinal agents. Optional components that are among those useful herein are disclosed in U.S. Pat. No. 4,387,090, the contents of which in this respect are herewith incorporated by reference.

Examples of suitable surfactants include, but are not limited to, Plantacare® 818 UP: Coco Glucoside, Texapon® NSO: Sodium Lauryl Ether Sulfate, Texapon® NSO: Sodium Lauryl Ether Sulfate, Dehyton® AB: Coco-betaine.

Examples of suitable emulsifiers include, but are not limited to, Xyliance (CETEARYL WHEAT STRAW GLYCOSIDES, CETEARYL ALCOHOL), Emulium® Delta (CETYL ALCOHOL, GLYCERYL STEARATE, PEG-75 STEARATE, CETETH-20, STEARETH-20).

Examples of suitable gelling agents include, but are not limited to, Natrosol™ 250HHR (Hydroxyethyl cellulose), Carbopol® ETD 2050 (Carbomer, XGF).

A wide variety of other additional components can be formulated into the present compositions and consumer products. These include: other conditioning agents, such as hydrolysed collagen, vitamin E, panthenol, panthenyl ethyl ether, hydrolysed keratin, proteins, plant extracts, and nutrients; hair-fixative polymers, such as amphoteric, non-ionic, cationic, and anionic fixative polymers, and silicone grafted copolymers; preservatives, such as benzyl alcohol, methyl paraben, propyl paraben, and imidazolidinyl urea; pH adjusting agents, such as glutamic acid, citric acid, sodium citrate, succinic acid, phosphoric acid, lactic acid, sodium hydroxide, and sodium carbonate; salts in general, such as potassium acetate and sodium chloride; coloring agents; hair oxidizing (bleaching) agents, such as hydrogen peroxide, perborate, and persulfate salts; hair reducing agents, such as thioglycolates; fragrances; and sequestering agents, such as disodium ethylenediamine tetra-acetate; ultraviolet and infrared screening and absorbing agents, such as octyl salicylate; and mixtures thereof.

The cosmetic composition of the present invention was found to provide a stimulation of sebum production, rendering it particularly suitable for the treatment of dry scalp and for the reduction of dry flakes on scalp.

In an embodiment of the present invention, the cosmetic composition is a scalp care composition.

The scalp care composition may be a leave-on or rinse-out product, for instance a shampoo, conditioner, spray, lotion, oil, gel, or wax.

The scalp care composition of the present invention may be applied to either wet or dry hair, depending on the formulation. Optionally, depending on the formulation, the hair may be rinsed, e.g. with water, after the application.

In an embodiment of the present invention, the cosmetic composition is a shampoo, an anti-dry dandruff product or a dry scalp lotion.

The cosmetic active ingredient of the present invention is particularly advantageously used in a product for dry skin and/or scalp.

The present invention further provides a method of stimulating the sebum production and/or of reducing the formation of dry flakes by applying the cosmetic active ingredient of the present invention or the cosmetic composition of the present invention to human scalp.

The present invention is further illustrated by means of the following non-limiting examples:

### EXAMPLE 1: ETHANOL/WATER EXTRACTION OF EXHAUSTED PATCHOULI AERIAL PARTS (COMPARATIVE Example)

Dried steam distilled patchouli aerial parts were crushed to obtain a powder. 75 g of powder were extracted at 20° C. in 550 g of ethanol (70% in water) for 60 min under stirring. The extract was filtered through a 0.7 μm cellulosic filter, concentrated five times to remove ethanol and finally filtered through a 0.35 μm cellulosic filter. The extract (1.078 g) was then freeze dried.

Fractionation of the Crude Extract by Centrifugal Partition Chromatography (CPC)

The crude extract (1.078 g) was dissolved in 30 ml a biphasic solvent system consisting of methyl-tert-butylether (MTBE), acetonitrile and water in a 3:3:4 ratio (v/v). Centrifugal Partition Chromatography (CPC) was performed using an FCPE300® instrument (Rousselet Robatel Kromaton) with a column of 303 ml, a column rotation speed of 1200 rpm and a flow rate of 20 ml/min.

An isocratic elution of the mobile phase (lower phase of the two-phase solvent system) was performed in the ascending mode for 75 min (with an initial flow rate ramp from 0 to 20 ml/min during 5 min). The column was finally extruded by switching the mode selection valve for 20 min. The CPC chromatogram was monitored at 220 nm. Fractions of 20 ml were collected over the whole experiment, and combined according to their thin layer chromatography (TLC) profiles. TLC was performed on pre-coated silica gel 60 F254 Merck plates with the migration solvent system EtOAc/toluene/formic acid/acetic acid (70/30/11/11; v/v), visualized under UV light at 254 nm and 360 nm and revealed by spraying the dried plates with 50% H2504 and vanillin followed by heating. As a result, 16 sub-fractions were collected.

NMR Analyses and Identification of the Major Metabolites

An aliquot of each fraction from F1 to F16 (up to ≈J 20 mg) was dissolved in 700 μl DMSO-d6 and analyzed by ¹³C NMR at 298 K on a Bruker Avance AVIII-600 spectrometer (Karlsruhe, Germany) equipped with a TXI cryoprobe. Spectra were manually phased and baseline corrected using the TOPSPIN 3.2 software (Bruker) and calibrated on the central resonance of DMSO-d6 (δ 39.80 ppm). The absolute intensities of all ¹³C NMR signals were automatically collected and binned across the spectra of the fraction series by using a locally developed computer script. The resulting table was imported into the PermutMatrix version 1.9.3 software (LIRMM, Montpellier, France) for Hierarchical Clustering Analysis (HCA). The resulting ¹³C chemical shift clusters were visualized as dendrograms on a two-dimensional map. For metabolite identification, each ¹³C chemical shift cluster obtained from HCA was manually submitted to the structure search engine of the database management software ACD/NMR Workbook Suite 2012 software, ACD/Labs, Ontario, Canada) comprising the structures and predicted chemical shifts of low molecular weight natural products (n≈J 2950 in March 2018). In parallel, a literature survey was performed to obtain the names and chemical structures of a maximum of metabolites already reported in the species Pogostemon cablin (n≈70). Additional 2D NMR experiments (HSQC, HMBC, and COSY) were performed on fractions containing putatively identified compounds in order to confirm the molecular structures proposed by the database at the end of the dereplication process.

The following major metabolites were identified:

| | |
|---|---|
| Verbascoside | Pachypodol |
| Apigenin-7-O-glucuronide | Cytosporone V |
| Succinic acid | p-hydroxybenzoic acid |
| Glucosyl-cytosporone V (2 isomers) | Protocatechuic acid |
| Lactic acid | Syringaresinol |
| Choline | Vanillin |
| α-D-fructose | Rhamnocitrin |
| β-D-fructose | Luteolin-7-O-glucuronide |
| α-D-glucose | Hydroxymethylglutaric acid |
| β-D-glucose | 12-hydroxyjasmonic acid |
| β-D-fructopyranose | a caffeoyl derivate |
| 7,3′-dimethyleriodictyol | apigenin-7-sugars |

The composition of the CPC fractions was as follows (Maj=major; Med=medium; Min=minor):

| CPC- | Mass | % crude | |
|---|---|---|---|
| Fractions | (mg) | extract | Composition |
| 1 | 7.9 | 0.7 | 7,3′-dimethyleriodictyol (Maj); Pachypodol (Maj); |
| Elution | | | Rhamnocitrin (Min) |
| 2 | 16.7 | 1.5 | 7,3′-dimethyleriodictyol (Maj); Pachypodol (Maj); |
| Elution | | | Cytosporone V (Med); Rhamnocitrin (Min) |
| 3 | 7.7 | 0.7 | p-hydroxybenzoic acid (Maj); Syringaresinol (Min); Vanillin |
| Elution | | | (Med) |
| 4 | 10.2 | 0.9 | Protocatechuic acid (Maj); Syringaresinol (Min); Vanillin |
| Elution | | | (Med) |
| 5 | 14.0 | 1.3 | 12-hydroxyjasmonic acid (Maj) + caffeoyl derivatives and |
| Elution | | | Apigenin-7-sugars |
| 6 | 11.8 | 1.1 | Apigenin-7-O-glucuronide (Min); Succinic acid (Min); caffeoyl |
| Elution | | | derivatives and Apigenin-7-sugars |
| 7 | 10.6 | 1.0 | Verbascoside (Min); Apigenin-7-O-glucuronide (Maj); |
| Elution | | | Succinic acid (Maj); Glucosyl-cytosporone V (Min) |
| 8 | 16.0 | 1.5 | Verbascoside (Med); Apigenin-7-O-glucuronide (Med); |
| Elution | | | Succinic acid (Maj); Glucosyl-cytosporone V (Min) |
| 9 | 26.7 | 2.5 | Verbascoside (Maj); Apigenin-7-O-glucuronide (Min); |
| Elution | | | Succinic acid (Med); Glucosyl-cytosporone V (Med); |
| | | | Luteolin-7-O-glucuronide (Min) |
| 10 | 26.9 | 2.5 | Verbascoside (Maj); Apigenin-7-O-glucuronide (Min); |
| Elution | | | Succinic acid (Min); Glucosyl-cytosporone V (Med); Luteolin- |
| | | | 7-O-glucuronide (Min); Hydroxymethylglutaric acid (Med) |
| 11 | 56.0 | 5.2 | Apigenin-7-O-glucuronide (Min); Succinic acid (Min); Lactic |
| Elution | | | acid (Maj); Luteolin-7-O-glucuronide (Min); |
| | | | Hydroxymethylglutaric acid (Maj) |
| 12 | 97.5 | 9.0 | Choline (Min); α-D-fructose (Min); β-D-fructose (Med); α-D- |
| Extrusion | | | glucose (Maj); β-D-glucose (Maj); β-D-fructopyranose (Min) |
| 13 | 329.4 | 30.3 | Choline (Min); α-D-fructose (Med); β-D-fructose (Maj); α-D- |
| Extrusion | | | glucose (Maj); ß-D-glucose (Maj); ß-D-fructopyranose (Med) |
| 14 | 306.0 | 28.2 | Choline (Min); α-D-fructose (Med); β-D-fructose (Maj); α-D- |
| Extrusion | | | glucose (Maj); ß-D-glucose (Maj); β-D-fructopyranose (Med) |
| 15 | 68.0 | 6.3 | Choline (Min); α-D-fructose (Med); β-D-fructose (Maj); α-D- |
| Extrusion | | | glucose (Maj); β-D-glucose (Maj); β-D-fructopyranose (Med) |
| 16 | 81.1 | 7.5 | Choline (Min); α-D-fructose (Min); β-D-fructose (Med); α-D- |
| Extrusion | | | glucose (Med); β-D-glucose (Med); β-D-fructopyranose (Min) |

### EXAMPLE 2: SUB-CRITICAL WATER EXTRACTION OF EXHAUSTED PATCHOULI AERIAL PARTS

Patchouli aerial parts Pogostemon cablin (Blanco) Benth were supplied by PT Indowangi Nusajaya (Indonesia).

The plants were cultivated in Sulawesi (Indonesia). Harvest takes place all year long. Patchouli aerial parts were dried during 3 days then gathered into bundles and kept a few days before distillation. This storage allows for a light fermentation process to release some key odour components of the plant. The patchouli aerial parts were then subjected to a first steam distillation locally by the farmers and to a subsequent redistillation in stainless steel vessels to obtain patchouli oil, leaving behind the exhausted patchouli aerial parts that can be used in the method of the present invention.

The exhausted aerial part of patchouli were dried under direct sunlight for at least 3 days (8 hours per day: 07.00-15.00). Additional drying time may be needed if it rains during the drying process. The ratio between leaves and stems is about 70%/30% (w/w).

Sub-Critical Water Extraction

For each production lot, between 0.75 kg and 1.1 kg of dried exhausted patchouli aerial parts were placed into a 5 l extraction chamber. Demineralized water is pumped through the extraction chamber by means of a high pressure pump (HPLC) at 150 ml/min until a pressure of 20 bar is reached. The water circulating through the extraction chamber is previously heated to a temperature of 125° C. by means of a heating resistor. The temperature is measured by means of a thermocouple (K-type) connected to a temperature control. At the exit of the extraction chamber, the extract is cooled using an ice bath and then collected in a vessel at atmospheric pressure.

In general, the extraction was performed at a ratio of plant to solvent of 1:5 (wt/wt), such that 12.75 kg of aqueous extract were obtained from 2.55 kg of plant, for instance.

2 kg of the thus obtained crude aqueous extract was diluted by the addition of 2 kg of demineralized water and filtered through a 1 μm filter cloth, followed by sterile filtration over a 0.2 μm filter plate to obtain 4 kg of an extract according to the present invention.

Fractionation

The sub-critical water extract was subjected to Centrifugal Partition Chromatography (CPC). Twelve fractions were obtained, each of which was analyzed by ¹³C NMR. It was found that fractions 10 to 12 contained 84.5% of the dry matter present in the crude extract.

The composition of the CPC fractions was as follows (Maj=major; Med=medium; Min=minor; recovery=97.2%):

| CPC- | Mass | % crude | |
|---|---|---|---|
| Fractions | (mg) | extract | Composition |
| 1 | 1.8 | 0.95 | Linear free fatty acids (Maj); 7,3′-dimethyleriodictyol (Med); |
| Elution | | | Pachypodol (Med) |
| 2 | 1.8 | 0.95 | Linear free fatty acids (Maj); 7,3′-dimethyleriodictyol (Med); |
| Elution | | | Pachypodol (Med) |
| 3 | 11.7 | 0.6 | Syringaresinol (Min); Cytosporone V (Maj); Linear free fatty |
| Elution | | | acids (Med); p-hydroxybenzoic acid (Min) |
| 4 | 15.8 | 0.8 | Protocatechualdehyde (Maj); Linear free fatty acids (Med); |
| Elution | | | Caffeic acid (Med); p-hydroxybenzoic acid (Maj); |
| | | | Protocatechuic acid (Med) |
| 5 | 22.2 | 1.2 | Vomifoliol (Med); Protocatechuic acid (Med); 12- |
| Elution | | | Hydroxyjasmonic acid (Med) |
| 6 | 30.4 | 1.6 | Succinic acid (Med); Apigenin-7-O-glucuronide (Maj) |
| Elution | | | |
| 7 | 82.5 | 4.3 | Succinic acid (Maj); 3-Hydroxy-3-methylglutaric acid (Min); |
| Elution | | | Apigenin-7-O-glucuronide (Med); Glucosyl-cytosporone V |
| | | | (Med); Luteolin-7-O-glucuronide (Min); Verbascoside (Min) |
| 8 | 32.1 | 1.7 | Succinic acid (Min); 3-Hydroxy-3-methylglutaric acid (Maj); |
| Elution | | | Apigenin-7-O-glucuronide (Min); Glucosyl-cytosporone V |
| | | | (Med); Lactic acid (Med); Pentonic acid 2-desoxy-y-lactone |
| | | | (Min); Luteolin-7-O-glucuronide (Med); Verbascoside (Min) |
| 9 | 30.5 | 1.6 | 3-Hydroxy-3-methylglutaric acid (Maj); Lactic acid (Maj); |
| Elution | | | Pentonic acid 2-desoxy-γ-lactone (Maj); Luteolin-7-O- |
| | | | glucuronide (Med) |
| 10 | 732.3 | 38.7 | Mixture of oliogosacchardies (not well soluble in DMSA-d6, |
| Extrusion | | | not fully identified); Glycerol |
| 11 | 758.6 | 40.0 | Mixture of oliogosacchardies (not well soluble in DMSA-d6, |
| Extrusion | | | not fully identified); Glycerol |
| 12 | 110.6 | 5.8 | Mixture of oliogosacchardies (not well soluble in DMSA-d6, |
| Extrusion | | | not fully identified); Glycerol (Maj) |

### EXAMPLE 3: DETERMINATION OF SUGAR CONTENT

The sugar content of the extract of the present invention, which was obtained by sub-critical water extraction, was compared to those of extracts prepared by classic extraction using water at high temperature and an ethanol/water mixture, respectively.

The extract of the present invention, Sample A, was prepared as described in Example 2 above.

The first comparative Sample B was prepared by extracting 40 g of exhausted patchouli aerial parts with 2×400 ml of water at reflux temperature for 2×2 h, followed by filtration over an AF6 Filtrox filter plate (15-35 μm) and over an AF31 H Filtrox filter plate (5-12 μm), using perlite as a filtration aid.

The second comparative Sample C was prepared as described in comparative Example 1 above.

The sugar content of the three samples was determined by high performance anion exchange chromatography with pulsed amperometric detection (HPAE-PAD) using calibration curves of rhamnose, arabinose, galactose, glucose, and mannose. To this end, 100 mg of each sample was placed into a 20 ml tube, and 2.5 ml of water and 0.5 ml of hydrochloric acid (37%) were added. The resulting mixture was mixed well and heated to 100° C. for 6 h. After cooling to reoom temperature, the mixture was transferred to a 10 ml volumetric flasked, which was filled up with water. 1 ml of the resulting diluted mixture was further diluted with 19 ml of water, passed through a 0.45 μm RC filter and an OnGuard Ag/H cartridge, before injection into the IC system.

| Extract | Total sugar content on a dry basis |
|---|---|
| Sample A | 45.6 wt % |
| Sample B | 14.9 wt % |
| Sample C | 8.42 wt % |

It was found that the extract of the present invention (Sample A) had a significantly higher total sugar content than the two conventional extracts, namely about triple for Sample B and more than five-fold for Sample C.

### EXAMPLE 4: COSMETIC ACTIVE INGREDIENT

495 g of the liquid sub-critical water extract obtained in Example 2 was diluted with 495 g of water, and 10 g of phenethyl alcohol was added. The thus obtained cosmetic active ingredient contained 1.2% of patchouli extract (based on dry matter), 0.94% of phenethyl alcohol, and 98.04% of water.

The thus obtained cosmetic active ingredient of the present invention is a water-soluble, brown to dark brown, liquid with a characteristic smell. It has a pH of 4-6, a Gardner Index of 16-18, a dry matter content of 1-5%, a phenethyl alcohol content of 0.9-1.2%, and a total sugar content of 0.07-0.4%. Microbiological specifications with regard to total plate count and yeast and mould count were both below 100 U.F.C./g.

### EXAMPLE 5: WOUND HEALING

Normal Human Keratinocytes (NHEKs) were seeded at 200′000 cells per well pre-coated with Type I collagen in 12-wells culture plates in the presence of keratinocyte growth medium (KGM, Lonza) supplemented with growth factors such as hydrocortisone, transferrin, epinephrine, bovine pituitary extract (BPE), recombinant human epidermal growth factor (rhEGF) and insulin. At confluence, cells were pre-incubated with the patchouli extracts and some of the fractions thereof described in examples 1 and 2 above, each at 0.5% (v/v), overnight in keratinocyte basal medium without supplement. After this pre-conditioning phase, the cell monolayer sticking to the bottom of the well was scratched with a P200 sterile cone, followed by two washes with phosphate buffered saline (PBS). The NHEK were then again stimulated with the same samples for 24 h in basal medium.

The wound healing process was analyzed by pictures recording at T₀ and T_{24 h} using inverting optical microscope (Zeiss). After image analysis, the percentage of scratch closing was determined relative to untreated condition.

Results are shown in the following table:

| | Scratch Closing [%] | |
|---|---|---|
| | Average | SEM |
| Untreated | 100 | 7 |
| Whole patchouli extract from example 1 | 2 | 1 |
| (comparative example) | | |
| Whole patchouli extract from example 2 | −1 | 1 |
| Fraction 2 from example 2 | 93 | 5 |
| Fraction 3 from example 2 | 100 | 18 |
| Fraction 4 from example 2 | 83 | 8 |
| Fraction 5 from example 2 | 86 | 26 |
| Fraction 6 from example 2 | 128 | 25 |
| Fractions 7-8 from example 2 | 75 | 4 |
| Fraction 9 from example 2 | 94 | 3 |
| Fractions 10-12 from example 2 | 17 | 7 |

As can be seen from the above, both whole patchouli extracts led to a complete inhibition of the keratinocytes migration. They are therefore both suitable for wound healing control. An inhibition of wound healing is particularly important in conditions involving a hyperproliferation of keratinocytes, e.g. hyperkeratosis or dry dandruff.

Within the patchouli extract according to the present invention, fractions 10-12 were found to be responsible for the inhibition of keratinocytes migration.

### EXAMPLE 6: ANTI-OXIDANT ACTIVITY

NHEKs were seeded in a black plate with a glass bottom at 20′000 cells per well pre-coated with type I collagen. Cells were incubated at 37° C. with 5% CO₂ for 24 h. On the next day, the cells were incubated for 24 h with the patchouli extracts and some of the fractions thereof described in examples 1 and 2 above, each at 0.5% (v/v), or Resveratrol (positive control) at 200 μM. After this pre-incubation, the cells were incubated with dichloro-dihydro-fluorescein (DCFH) probe at 50 μM for 30-45 min. The cells were then rinsed two times with PBS and incubated with PBS alone or with PBS containing 5 mM tert-butyl peroxide (TBP) to induce an oxidative stress. Fluorescence reading was performed every 10 min for 1 h, exciting at 488 nm and emitting at 525 nm.

The results are shown in the following table:

| | | ROS Production [% of | |
|---|---|---|---|
| | | TBP, untreated] | |
| | | Average | SEM |
| No TBP | Untreated | 32 | 2.7 |
| TBP | Untreated | 100 | 4.8 |
| | Resveratrol | 18 | 1.1 |
| | Patchouli Extract of Example 1 | 69 | 3.4 |
| | Patchouli Extract of Example 2 | 51 | 4.0 |
| | Fractions 10-12 of Example 2 | 77 | 3.3 |

As can be seen from the above, the whole patchouli extract of example 2 led to a reduction in reactive oxygen species (ROS) production by −49% while the comparative whole patchouli extract of example 1 showed a reduction of ROS by only −31%. Thus, the extract of the present invention is significantly more efficient than the comparative sample.

Interestingly, it was found that the fractions 10-12 were also involved in the antioxidant property of the patchouli extract.

### EXAMPLE 7: CLINICAL STUDY 1: REACTIVATION OF SEBUM PRODUCTION ON DRY SCALP BY APPLICATION OF a Shampoo (Rinse-Off)

This first clinical study was performed to evaluate the efficacy of the cosmetic active ingredient of the present invention in a rinse-off application on volunteers having dry scalp, itching, scalp discomfort and white flakes. The patchouli extract was tested in a shampoo at a concentration of (v/v) versus a placebo. The volunteers applied the shampoo every other day for 28 days.

INCI Formula and Composition of Shampoos
Placebo: AQUA/WATER, SODIUM LAURETH SULFATE, COCAMIDOPROPYLBETAINE, SODIUM CHLORIDE, PHENOXYETHANOL, FRAGRANCE
Active: AQUA/WATER, SODIUM LAURETH SULFATE, COCAMIDOPROPYLBETAINE, PATCHOULI EXTRACT, SODIUM CHLORIDE, PHENOXYETHANOL, FRAGRANCE

| | Placebo (v/v) | Active (v/v) |
|---|---|---|
| AQUA/WATER | 57.5% | 57.0% |
| SODIUM LAURETH SULFATE | 28.9% | 28.9 |
| COCAMIDOPROPYLBETAINE | 12.0% | 12.0% |
| SODIUM CHLORIDE | 0.5% | 0.5% |
| PHENOXYETHANOL | 0.6% | 0.6% |
| FRAGRANCE | 0.5% | 0.5% |
| PATCHOULI EXTRACT | — | 0.5% |

Panel Description

A single-center study with 40 volunteers exhibiting dry and itchy scalp was carried out. Volunteers were divided in two groups of 20 volunteers as following:
Group 1:20 volunteers in total, including 10 women and 10 men with an average age of years
Group 2: 20 volunteers in total, including 14 women and 6 men with an average age of 51±11 years

The study was conducted according to the standard operating procedure of Centro de Tecnologia Capilar S.L. and in compliance with the regulations established in “Gula para investigaciones con seres humanos” (Guidelines for Research on Human Beings) and the guidelines of the Scientific Committee on Consumer Safety (SCCS).

The volunteers applied the active (containing 0.5% of the extract of example 2 above) or placebo shampoo every other day for 28 days.

Statistical Analysis

For all clinical studies described herein, the following statistical analysis was performed:
Comparison before/after:
   a Shapiro-Wilk test was performed to determine the normality of differences (α=0.05)
   according to the result of the normality tested, either a paired Student t test or a Wilkoxon signed rank test was performed
   results with p≥0.05 were rejected as not statistically significant
   results with p≤0.05 were considered statistically significant
Comparison product/placebo:
   a Shapiro-Wilk test was performed to determine the normality of data within each group (α=0.05)
   according to the result of the normality tested, either an unpaired Student t test or a Mann-Whitney test was performed
   results with p≥0.05 were rejected as not statistically significant
   results with p≤0.05 were considered statistically significant
For the analysis of self-assessment results, a Chi-square test was done (dichotomous analysis which consists in comparing the number of associated answers)

Sebum Analysis by Sebufix® Scoring

Scalp sebum is measured at different times to assess whether the tested products have sebum regulating properties. In this case, the SEBUFIX® (Sebufix®, CKelectronic, Cologne, Germany) is applied to the upper part of the scalp to achieve the absorption of the sebum present on the scalp. The microcamera is a professional diagnostic piece of equipment consisting of a probe for exploring the scalp and the software for capturing and displaying images. The microcamera's function is to allow a direct, magnified view of a scalp area to observe its state: whether flakiness, greasiness or redness are present, etc.

The attributes under study—dandruff, redness, greasiness, etc.—were evaluated on a scale of 0-5: The lowest value (0) corresponds to the level “absence” of the attribute and the highest value (5) corresponds to the level “very high”. The intermediate values 1, 2, 3 and 4 correspond to the levels “low”, “average”, “considerable” and “high”, respectively.

Results

After 28 days of shampoo application, it was observed that the placebo shampoo led to a decrease in sebum by −25% compared to D₀, while the shampoo containing 0.5% of the patchouli extract of example 2 led to a slight increase by +9%.

In addition, a significant improvement of the sebum production was observed with the active by +34% in comparison to the placebo.

These effects were also confirmed by illustrative pictures showing lipid droplets caused by sebum on Sebufix®: Indeed, an increase of lipid droplets was observed for the active, while the placebo led to a decrease, as observed by the distribution of dark spots on illustrative pictures.

These results demonstrate that the patchouli extract of the present invention is able to reactivate sebum production on dry scalp.

### EXAMPLE 8: CLINICAL STUDY 2: IMPACT ON SCALP MICROBIOTA COMPOSITION ON DRY SCALP BY Application of a Shampoo (Rinse-Off)

Microbiota Sampling and Storage

Two groups of 20 volunteers each (same panel as in example 7), exhibiting dry and itching scalp presenting white flakes, had to apply on their scalp and hair a shampoo formula either containing the active (vehicle+patchouli extract of example 2 at 0.5%) or a placebo (vehicle only), every other day for 28 days.

Scalp samples of microflora were collected from the occiput by a non-invasive swabbing method using sterile swabs moistened with a sterile solution of 0.15 M NaCl. Swabs were transferred at −20° C. and kept frozen until DNA extraction. Samples were taken before treatment (D₀), and after 28 days of treatment (D₂₈), using a standardized procedure.

DNA Extraction

DNA extraction was performed, for 92 samples (2×20 test samples plus 12 negative control samples), using the DNeasy PowerLyzer® PowerSoil® DNA Isolation Kit with Qiacube device (Qiagen, Hilden, Germany), with the following modifications: The tip of each swab was detached with a sterile surgical blade and transferred into a 1.5 ml tube containing 750 μl of Bead Solution. The sampled biomass was suspended by stirring and pipetting, and then transferred to a bead beating tube. The remaining steps were performed according to the manufacturer instructions. DNA concentration was determined using the QuBit dsDNA HS fluorometric quantitation kit (Invitrogen, ThermoFisher Scientific, Courtaboeuf, France) according to the manufacturer's instructions.

Sequencing and Data Analysis

16S rRNA Gene Sequencing

Sequencing was performed using a MiSeq device (Illumina, Inc., San Diego, CA, USA) through a 500 cycles paired-end run, targeting the V3V4 16S variable regions using the following primers: 16S-Mi341F forward primer 5′-CCTACGGGNGGCWGCAG-3′ and 16S-Mi805R reverse primer producing about 460 bp amplicons.

PCR1s were performed as follows: 8 μl of template DNA (0.2 ng) were mixed with 5 μl of each reverse and forward primers (1 μM), 5 μl of KAPA HiFi Fidelity Buffer (5×), 0.8 μl of KAPA dNTP Mix (10 mM each), 0.7 μl of RT-PCR grade water (Ambion), and 0.6 μl of KAPA HiFi hotstart Taq (1 U/μl), for a total volume of 25 μl. Each amplification was duplicated, and duplicates were pooled after amplification. PCR1 cycles consisted of 95° C. for 3 min and then 32 cycles of 95° C. for 30 s, 59° C. for 30 s, and 72° C. for 30 s, followed by a final extension at 72° C. for 3 min, with a BioRad CFX1000 thermocycler. Negative and positive controls were included in all steps to check for contamination. All duplicate pools were controlled by gel electrophoresis, and amplicons were quantified using fluorometry.

Libraries ready for analysis were then produced following the Illumina guidelines for 16S metagenomics libraries preparation. Briefly, the PCR1 amplicons were purified and controlled using an Agilent 2100 Bioanalyzer (Agilent Technologies, Santa Clara, USA). To enable the simultaneous analysis of multiple samples (multiplexing), Nextera® XT indexes (Illumina) were added during PCR2 using between 15 to 30 ng of PCR1 amplicons. PCR2 cycles consisted of 94° C. for 1 min and then 12 cycles of 94° C. for 60 s, 65° C. for 60 s, and 72° C. for 60 s, followed by a final extension at 72° C. for 10 min. Indexed libraries were purified, quantified and controlled using an Agilent 2100 Bioanalyzer. Validated indexed libraries were pooled in order to obtain an equimolar mixture.

The run (500 cycles) was achieved on MiSeq sequencer (Illumina) using the MiSeq Reagent Kit v3 600 cycles (Illumina). The sequencing run produced an output of 12.7 million of paired-end reads of 250 bases, i.e. up to 3.2 Gigabases.

After the MiSeq run, raw data sequences were demultiplexed and quality-checked to remove all the reads with ambiguous bases. Indexes and primers sequences were removed with cutadapt (v1.9; http://cutadapt.readthedocs.io/en/stable/index.html) and reads with fastq score lower than 28 were trimmed. The forward and reverse sequences were paired using bbmerge (https://jgi.doe.gov/data-and-tools/bbtools/). Samples with less than 5000 paired-sequences were discarded. The remaining paired sequences were then treated using an in-house pipeline that uses vsearch (Rognes et al., 2016) to remove chimeras and amplicons with PCR errors. Sequences were then split into Operational Taxonomic Units (OTUs, a cluster of similar sequence variants of the 16S rRNA marker gene sequence) at a 1% dissimilarity level using swarm (v2.6—Mahé et al., 2015). Unique amplicons were mapped to the SILVA SSU Ref NR 99 (non-redundant) database (release 132; https://www.arb-silva.de/) for taxonomic assignation using the RDP classifier (Wang et al., 2007). Data normalization and analyses were done using R statistical computing environment (v3.2.0; https://www.r-project.org/—R core team (2014) using Bioconductor package (mainly Phyloseq, DESeq2 and Vegan libraries; http://www.bioconductor.org/).

Data were then compared using Wilcoxon's test for paired samples (Wilcoxon, 1945). Due to multiple testing, p-value were adjusted using false discovery rate (FDR) correction (Binyamini and Hochberg, 1995)

Results

The impact of sebum level on scalp microbiota composition was studied using a 16S sequencing approach. A significant variation on scalp microbiota composition was observed, which is related to the amount of sebum. More particularly, an important increase of the Cutibacterium abundance was observed on oily scalp with +54%. The high dominance of Cutibacterium on oily scalp would explain the decrease of alpha diversity through the selective pressure mediated by the increased quantity of sebum. Other significant modifications were also observed, such as a strong decrease of Corynebacterium, Streptococcus, Micrococcus and Kocuria by −71%, 21%, −33% and 74%, respectively.

This shows that a high abundance of Cutibacterium can be used as marker to evaluate the impact of sebum on the scalp microbiota composition. Indeed, a dry scalp presents a low level of sebum linked to an important alpha diversity and a low abundance of Cutibacterium.

During the clinical evaluation of the patchouli extract, scalp microbiota samples were collected by swabbing scalps in order to assess the improvement of dry scalp.

After treatment with the active, a decrease in alpha diversity (Shannon Index) by 19.5% was observed, while the placebo treatment led to a decrease by only 4.4%. Thus, the patchouli extract of the present invention induces the sebum production on dry scalp and triggers a decrease of microbiota diversity.

Furthermore, after placebo treatment, an important decrease of Cutibacterium proportion by −18.5% was observed after 28 days of application, while the application of the active led to a stabilising of the Cutibacterium proportion (slight increase by +3.2%). Thus, the patchouli extract of the present invention increased the abundance of Cutibacterium on dry scalp, which is an indicator for a better scalp condition.

Overall, the above results demonstrate that the patchouli extract of the present invention is able to increase the sebum production on dry scalp, to decrease the alpha diversity, and to increase the Cutibacterium abundance. These observations clearly indicate that the extract is suitable for treating dry scalp condition.

### EXAMPLE 9: CLINICAL STUDY 3: REDUCTION OF WHITE FLAKES ON DRY SCALP BY APPLICATION OF A Shampoo (Rinse-Off)

INCI Formula and Composition of Shampoos
Placebo: AQUA/WATER, SODIUM LAURETH SULFATE, COCAMIDOPROPYLBETAINE, SODIUM CHLORIDE, PHENOXYETHANOL, FRAGRANCE
Active: AQUA/WATER, SODIUM LAURETH SULFATE, COCAMIDOPROPYLBETAINE, PATCHOULI EXTRACT, SODIUM CHLORIDE, PHENOXYETHANOL, FRAGRANCE

| | Placebo (v/v) | Active (v/v) |
|---|---|---|
| AQUA/WATER | 57.5% | 57.0% |
| SODIUM LAURETH SULFATE | 28.9% | 28.9 |
| COCAMIDOPROPYLBETAINE | 12.0% | 12.0% |
| SODIUM CHLORIDE | 0.5% | 0.5% |
| PHENOXYETHANOL | 0.6% | 0.6% |
| FRAGRANCE | 0.5% | 0.5% |
| PATCHOULI EXTRACT | — | 0.5% |

Panel Description

This third clinical study was carried out on 41 volunteers aged over 18 years, exhibiting dry and itchy scalp with the presence of white flakes. The volunteers were divided in two groups:
Group 1: 21 volunteers including 5 men and 16 women with an average age of 33±2 years
Group 1: 20 volunteers including 4 men and 16 women with an average age of 33±1 years

They willingly signed the consent form which was written in compliance with the Declaration of Helsinki and the Dec. 20, 1988 act of the Code de la Santé Publique.

During this study, volunteers applied a shampoo containing 0.5% of active or placebo every two days for 28 days.

White Flakes Reduction Analysis by Scoring

For the clinical evaluation, four regions of the head were assessed separately: front left, front right, back left, and back right. Scores were then calculated as the mean of the four regions for each volunteer.

Two days after application of the shampoo, non-adherent dandruff was evaluated on a scale of 0-5: The lowest value (0) corresponds to “no dandruff”, and the highest value (5) corresponds to “a very large quantity of dandruff”. The intermediate values 1, 2, 3 and 4 correspond to “a few dispersed dandruff flakes”, “a small quantity of dandruff”, “a moderate quantity of dandruff” and “a large quantity of dandruff”, respectively.

It was found that the shampoo containing the patchouli extract of example 2 at 0.5% significantly reduced the number of white flakes by 23% and 33% after 14 and 28 days of application, respectively.

By comparison, the application of the placebo shampoo only slightly reduced the number of white flakes by 9% after 14 days, and the reduction was only significant after 28 days of application, showing a reduction by 20% compared to Do.

The difference between the active of the present invention and the placebo was found to be significant.

These results demonstrate that the patchouli extract of the present invention is able to significantly reduce white flakes on dry scalp in a rinse-off application (shampoo).

Illustrative Pictures Taken by C-Cube®

C-Cube 2® (Pixience) dermoscope allows skin image acquisition in Ultra High Definition (4K UHD video streaming; 18 million pixels image resolution). It incorporates True Color patented technology, which optimally renders the full spectrum of the skin's natural colours. The size of image acquisition is 12×16 mm and the magnification is ×5.

It was found that the application of placebo shampoo for 28 days had only a very slight effect on white flakes, while the shampoo containing the patchouli extract at 0.5% was efficient drastically reduced white flakes.

### EXAMPLE 10: CLINICAL STUDY 4: IMPACT ON DRY SCALP AND WHITE FLAKES USING A LEAVE-ON PRODUCT (Hair Lotion)

INCI Formula and Composition of Hair Lotions
Placebo: AQUA/WATER, SODIUM BENZOATE, PPG-26 BUTETH-26, PEG-40 HYDROGENATED CASTOR OIL, CITRIC ACID, FRAGRANCE, CITRONELLOL, BUTYLPHENYL, METHYLPROPIONAL, D-LIMONENE, ALPHA-ISOMETHYL IONONE
Active: AQUA/WATER, PATCHOULI EXTRACT, SODIUM BENZOATE, PPG-26 BUTETH-26, PEG-40 HYDROGENATED CASTOR OIL, CITRIC ACID, FRAGRANCE, CITRONELLOL, BUTYLPHENYL, METHYLPROPIONAL, D-LIMONENE, ALPHA-ISOMETHYL IONONE

| | Placebo (v/v) | Active (v/v) |
|---|---|---|
| AQUA/WATER | 99.1% | 98.6% |
| SODIUM BENZOATE | 0.5% | 0.5% |
| PPG-26 BUTETH-26, PEG-40 | 0.2% | 0.2% |
| HYDROGENATED CASTOR OIL, | | |
| WATER (Solubilizing LRI solution) | | |
| CITRIC ACID | 0.1% | 0.1% |
| FRAGRANCE, CITRONELLOL, | 0.08% | 0.08% |
| BUTYLPHENYL, | | |
| METHYLPROPIONAL, D-LIMONENE, | | |
| ALPHA-ISOMETHYL IONONE | | |
| PATCHOULI EXTRACT | — | 0.5% |

Panel Description

This fourth clinical study was done in a double-blind, randomized and placebo controlled setting. The assessment was based on an intra-subject comparison.

All subjects received verbal and written information concerning the study. This information emphasized that participation in the study is voluntary and that the subject may withdraw from the study at any time and for any reason. All subjects were given the opportunity to ask questions about the study and were given sufficient time to consider their participation before consenting. The subject's written informed consent to participate in the study was obtained prior to any study-related procedure being carried out.

30 volunteers, aged between 18 and 75, with a mean age of 43.4±13.3 years, where split into two groups of 15 each. Volunteers were exhibiting white flakes, itching on the scalp and with a dry scalp (Corneometer®<50 a.u on the forehead/scalp).

Volunteers applied the respective products twice a day for 28 days to their scalp according to a 12-point pattern (3 lines of 4 points each on left, middle and right, going from front to back). After application of the products, the application points were massaged until full product penetration.

The reduction of white flakes was evaluated by scoring and self-assessment.

White Flakes Reduction Analysis by Scoring

Evaluation of the amount of white flakes was done by scoring using the following scale from 1 to

| Grade 1 | Grade 2 | Grade 3 | Grade 4 | Grade 4 |
|---|---|---|---|---|
| No presence | No visible dry | Some visible dry | Important visible | Important dry |
| of dry | dandruffs but | dandruffs and | dry dandruffs and | dandruffs on scalp |
| dandruffs | visible after | visible after | visible after | and clothes without |
| | scratching | scratching | scratching | and with scratching |

After 28 days of application of the active, a significant reduction of white flakes by 21% was observed compared to Do.

The application of the placebo formula, on the other hand, led to a slight but not significant increase of white flakes by 10%.

Thus, a significant reduction of white flakes by −31% with the active lotion compared to the placebo lotion was observed after 28 days of application.

These results demonstrate the strong efficacy of the extract of the present invention on the reduction of white flakes from dry and itching scalp.

Self-Assessment

After 28 days of application, only 13% of volunteers applying the active lotion had intense and frequent itching, compared to 47% of those applying the placebo lotion. These differences were significant, demonstrating that the lotion containing the patchouli extract of the present invention significantly reduces the frequency and intensity of itching in comparison with placebo.

It was further observed that only 7% of volunteers who applied the active lotion found white flakes on their clothes, compared to 33% of those applying the placebo lotion. This observation demonstrates that the cosmetic active ingredient of the present invention is able to significantly reduce the white flakes found on clothes in comparison to the placebo effect.

### EXAMPLE 11: CLINICAL STUDY 5: EMOTIONAL EVALUATION BY NEUROSCIENCE METHODS (LEAVE-ON Product)

This fifth clinical study was performed in the same setting as the fourth clinical study described in example 12 above.

Prosody Analysis

The participants were asked to verbalize how they feel about their scalp at the beginning and end of the 28-day study. From the audio recording of their free verbal productions, a prosody analysis was applied. The prosody analysis consists in capturing the vocal signal of the verbal expression of subjects and analyzing the physical parameters of the voice in terms of emotional expression.

Using computer-based solutions, two main variables were extracted:
Loudness (i.e. vocal intensity), represented by the mean amplitude measured in dB
Pitch (i.e. tone), represented by the coefficient of variation of the fundamental frequency (cvFO) measured in Hz)

The combination of these two variables allows for assessing the degree of emotional valence and arousal expressed vocally. An increase of frequency and amplitude in this context suggest that volunteers felt more positive emotions.

It was found that, after 28 days of application, the active lotion led to a stronger increase in the frequency and amplitude than the placebo lotion. Furthermore, the active showed a significant increase of frequency and amplitude in comparison with placebo by +15.4% and +12.4% respectively.

Wherefore, the extract of the present invention was able to significantly improve the emotional response after 28 days of application, as observed by prosody evaluation.

In order to complete this analysis, an emotional valence analysis was performed, permitting to study the emotional distribution considering stimulating/unstimulating and pleasant/unpleasant emotions scale.

It was found that the active lotion triggered stimulating and pleasant emotions, while the placebo lotion triggered neutral emotions.

Finally, the percentage of volunteers who have triggered negative and positive emotions was analysed for each group. It was found that volunteers applying the active lotion have triggered 80% of positive emotions and 20% negative ones. In contrast, volunteers who applied the placebo lotion, have triggered only 33% of positive emotions and 66% negative ones.

Mood Portraits®

The Mood Portraits® method was used to measure the emotional responses of consumers at the beginning and end of the 28-day study. Participants were asked to think about the condition of their current scalp and the emotions that this brought to their minds. They were then asked to select pictures representing these emotions.

Day 0 results showed that participants had a very negative emotional response when thinking about their scalp. Day 28 results showed that participants had a significantly more positive emotional response when thinking about their scalp, with the active group evoking more significant positive mood responses than the placebo.

On D₀, participants felt very negative, and significantly not happy, when thinking about their scalp.

On D₂₈, participants in the active group felt significantly less negative after using the hair lotion for 28 days and significantly more refreshed.

Participants in the placebo group did not feel as negatively about their scalp on D₂₈; however, there was no significant difference. No positive mood was significantly evoked, but participants tended to feel more refreshed.

There is a significant difference between the active group, the placebo group and the D₀ results for refreshed and negative moods.

This effect was also visible through the images selected by volunteers at D₀ and D₂₈: At Do, volunteers illustrated their feelings about their scalp condition by images having negative and non-happy connotations.

After 28 days of active lotion application, a strong decrease of negative images associated was observed, as well as new images with a connotation of refreshing and self-confidence.

The group who applied placebo did not show the same efficacy, as shown by the selected images which are still negative.

This results show that the patchouli extract of the present invention delivers a positive emotional response after 28 days of application on volunteers who have dry itching scalp with white flakes. The improvement of the volunteers' mood is correlated to a significant reduction of white flakes and itching and, thus, to an improvement of their scalp condition.

Non-Verbal Communication Using Non-Verbal Decoding System®

Measurement of the unconscious emotional power of the lotion on the consumers was carried out by Marina Cavassilas of Semiopolis, an expert in non-verbal communication. To determine the emotional impact of the tested lotions, the body language of 14 users who used the active lotion, compared to that of 15 users who used the placebo.

The participants were interviewed on their use of the lotion and filmed face to face for about 5 minutes. A study of their expressiveness had been organized before the study to check their normal abilities to express their emotions by the face. This methods uses a unique grid to analyse over 200 non-verbal reactions (facial reactions, postures, gestures, voice), and remove all verbalisation barriers.

After 28 days of active lotion application, it was observed that 19 positive emotions and only 4 negative emotions were triggered in the volunteers. In contrast, the volunteers applying placebo lotion presented an inverse emotional response, with 14 negative emotions and only 2 positive emotions after 28 days.

These results demonstrate that the patchouli extract of the present invention was able to deliver a positive emotional response compared to the placebo lotion. Interestingly, it was found that this difference is statistically significant.

This method permits also to identify the type emotions after product application.

It was observed that volunteers who applied the active lotion showed positive emotions including comfort, care and happiness, while no any specific emotions were attributed to the placebo lotion. In addition, some emotions were found to be common to both the active and the placebo, including vigor, energy, power, and annoyance.

Thus, using non-verbal communication, it was found that the patchouli extract delivered a positive emotional response after 28 days of application on volunteers who have dry itching scalp with white flakes.

1. A method of preparing a cosmetic active ingredient, said method comprising the steps of:
a) providing exhausted patchouli aerial parts; and
b) performing a sub-critical water extraction on the exhausted patchouli aerial parts.

2. The method of claim 1, wherein sub-critical water extraction is performed at a temperature of 100 to 175° C., and a pressure of 1 to 50 bar.

3. The method of claim 1, further comprising the step of:
c) solid-liquid separation.

4. The method of claim 1, further comprising the step of:
d) adding a preservative, preferably phenethyl alcohol.

5. The method of claim 1, further comprising the step of:
e) sterilization.

6. A cosmetic active ingredient obtained by the method of claim 1.

7. The cosmetic active ingredient of claim 6, wherein the cosmetic active ingredient comprises a total sugar content of about 3-15 wt % on a dry basis.

8. A cosmetic composition comprising the cosmetic active ingredient of claim 6 and a carrier.

9. The cosmetic composition of claim 8, wherein the cosmetic composition is a scalp care composition.

10. The cosmetic composition of claim 9, wherein the cosmetic composition is a shampoo, an anti-dry dandruff product or a dry scalp lotion.

11. A method of stimulating the sebum production and/or of reducing the formation of dry flakes by applying the cosmetic active ingredient according to claim 6 to human scalp.

12. The method of claim 2, wherein the sub-critical water extraction is performed at a temperature of 110 to 150° C.

13. The method of claim 12, wherein the sub-critical water extraction is performed at a temperature of about 125° C.

14. The method of claim 2, wherein the sub-critical water extraction is performed at a pressure of 10 to 30 bar.

15. The method of claim 14, wherein the sub-critical water extraction is performed at a pressure of about 20 bar.

16. The method of claim 3, further comprising the step of:
d) adding a preservative.

17. The method of claim 4, wherein the preservative is phenethyl alcohol.

18. The method of claim 4, further comprising the step of:
e) sterilization.

19. The method of claim 5, comprising the step of:
e) sterile filtration.

20. The cosmetic active ingredient of claim 7, wherein the cosmetic active ingredient comprises a total sugar content of at least 10 wt %.

21. A method of stimulating the sebum production and/or of reducing the formation of dry flakes by applying the cosmetic composition according to claim 8 to human scalp.
